(19) United States
(12) Patent Application Publication
(10) Pub. No.: US 2022/0369939 A1
(43) Pub. Date: Nov. 24, 2022

(54) BLOOD FLOW OCCLUSION DEVICES AND METHODS THEREOF

(71) Applicant: George Logan DAUGHTRY, Charlotte, NC (US)

(72) Inventor: George Logan DAUGHTRY, Charlotte, NC (US)

(21) Appl. No.: 17/749,323

(22) Filed: May 20, 2022

Related U.S. Application Data
(60) Provisional application No. 63/191,140, filed on May 20, 2021.

(51) Int. Cl. A61B 5/0225; G16H 10/60; A61B 5/022; A61B 5/024; A61B 5/026; A61B 5/00

(52) U.S. Cl. CPC A61B 5/0225; G16H 10/60; A61B 5/02233; A61B 5/024; A61B 5/026; A61B 5/742; A61B 5/7475

(57) ABSTRACT

A blood flow occlusion system includes an inflator and a pressure regulator coupled to a pressure cuff and a computing device coupled to the inflator and the regulator. The computing device comprises a memory coupled to the processor which is configured to be capable of executing programmed instructions stored in the memory to: determine brachial occlusion data based on calculating the brachial occlusion data with an occlusion regression equation using one or more input diagnostic parameters associated with a client; generate treatment parameters of a treatment program to treat the condition of the client based on the optimized brachial occlusion data and an obtained stage of the condition; generate programmed instructions of control commands to manage operation of the pressure cuff, inflator, and pressure regulator based on the treatment parameters; and initiate execution of the programmed instructions for the treatment program for treating the condition of the client when engaged.

This application claims the benefit of Provisional Patent Application Ser. No. 63/191,140, filed May 20, 2021, which is incorporated by reference in its entirety.

### FIELD

This technology relates to devices and methods for blood flow occlusion for different applications, such as managing blood pressure.

### BACKGROUND

High blood pressure, also referred to as hypertension, is a common medical condition in which a high long-term force of blood against walls of the arteries may eventually cause health problems, such as heart disease. Typically, initial attempts to treat high blood pressure involve changes in lifestyle, relating to adjustments to diet and exercise. Unfortunately, in many cases these lifestyle changes are insufficient and as a result treatment of the high blood pressure requires the use of medications which have their own side effects and limitations.

### SUMMARY

A blood flow occlusion system includes an inflator and a pressure regulator coupled to a pressure cuff and a programmable controller computing device coupled to the inflator and the pressure regulator. The computing device comprises a memory coupled to the processor which is configured to be capable of executing programmed instructions stored in the memory to: determine brachial occlusion data based on calculating the brachial occlusion data with an occlusion regression equation using one or more input diagnostic parameters associated with a client; generate one or more treatment parameters of a treatment program to treat the condition of the client based on at least the brachial occlusion data and an obtained stage of the condition for the client; generate programmed instructions of control commands to manage operation of the pressure cuff, the inflator, and the pressure regulator based on the one or more treatment parameters of the treatment program; and initiate execution of the programmed instructions of the control commands for the determined treatment program for treating the condition of the client when engaged.

A method for making a blood flow occlusion system includes providing a pressure cuff and coupling an inflator and a pressure regulator to the pressure cuff. A programmable controller computing device is coupled to the inflator and the pressure regulator and comprising a memory coupled to the processor which is configured to be capable of executing programmed instructions stored in the memory to: determine brachial occlusion data based on calculating the brachial occlusion data with an occlusion regression equation using one or more input diagnostic parameters associated with a client; generate one or more treatment parameters of a treatment program to treat the condition of the client based on at least the brachial occlusion data and an obtained stage of the condition for the client; generate programmed instructions of control commands to manage operation of the pressure cuff, the inflator, and the pressure regulator based on the one or more treatment parameters of the treatment program; and initiate execution of the programmed instructions of the control commands for the determined treatment program for treating the condition of the client when engaged.

A non-transitory computer readable medium having stored thereon instructions comprising executable code that, when executed by one or more processors, causes the one or more processors to: determine brachial occlusion data based on calculating the brachial occlusion data with an occlusion regression equation using one or more input diagnostic parameters associated with a client; generate one or more treatment parameters of a treatment program to treat the condition of the client based on at least the brachial occlusion data and an obtained stage of the condition for the client; generate programmed instructions of control commands to manage operation of the pressure cuff, the inflator, and the pressure regulator based on the one or more treatment parameters of the treatment program; and initiate execution of the programmed instructions of the control commands for the determined treatment program for treating the condition of the client when engaged.

This technology provides a number of advantages including providing blood occlusion devices and methods for different applications including assisting with conditions, such as managing blood pressure by way of example. With examples of this technology, resting blood pressure is reduced and hypertension is effectively treated through a passive, non-pharmacological means.

### BRIEF DESCRIPTION OF THE DRAWINGS

FIG. 1 is a block diagram of an example of a blood flow occlusion system.

FIG. 2 is a flow chart of an example of a method for managing a condition with a blood flow occlusion system.

FIG. 3 is an example of a conversion table used in generating a treatment program for managing a condition.

### DETAILED DESCRIPTION

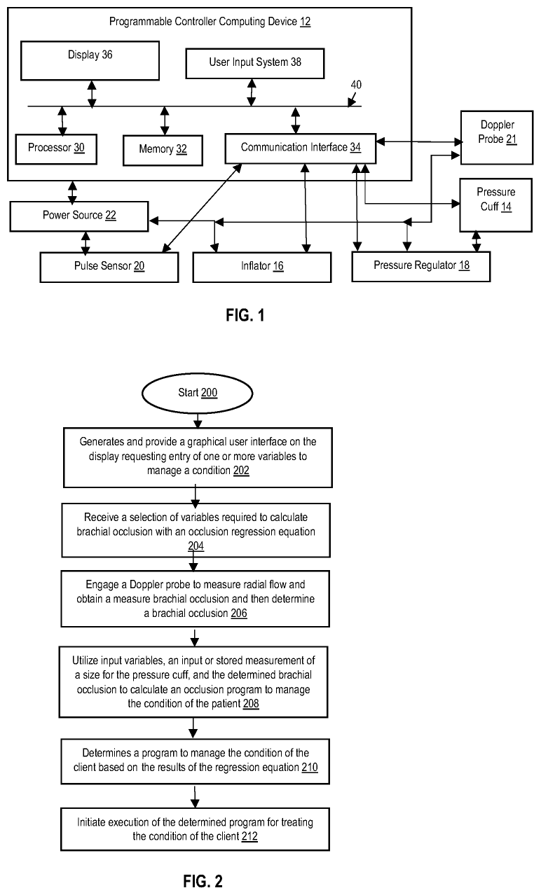

An exemplary blood flow occlusion system 10 is shown in FIG. 1. In this example, the blood flow occlusion system 10 includes a programmable controller computing device 12, a pressure cuff 14, an inflator 16, a pressure regulator 18, a pulse sensor 20, a Doppler probe 21, and a power source 22, although the system may have other types and/or numbers of other systems, devices, components, and/or other elements in other configurations. This technology provides a number of advantages including providing blood occlusion devices and methods for different applications including assisting with conditions, such as managing blood pressure by way of example.

Referring more specifically to FIG. 1, the programmable controller computing device 12 includes a processor 30, a memory 32, a communication interface 34, a display device 36, and a user input system 38 which are coupled together by a bus or other link 40, although other types of control systems may be used and the device may have other types and/or numbers of other systems, devices, components, and/or other elements.

The processor 30 of the programmable controller computing device 12 may execute programmed instructions stored in the memory 32 for one or more aspects of the present technology as illustrated and described by way of the examples herein. The processor 30 of the programmable controller computing device 12 may include one or more CPUs or general purpose processors with one or more processing cores, for example, although other types of processor(s) can also be used.

The memory 32 of the programmable controller computing device 12 stores these programmed instructions for one or more aspects of the present technology as illustrated and described by way of the examples herein, although some or all of the programmed instructions could be stored elsewhere. In this example, the memory 32 of the programmable controller computing device 12 stores one or more applications that can include computer executable instructions that, when executed by the programmable controller computing device 12, cause the programmable controller computing device 12 to perform actions, such as to therapeutically manage blood occlusion as illustrated and described by way of the examples herein. The memory 32 may also store the exemplary occlusion regression equation for a 5 cm pressure cuff 14 and a conversion table shown in FIG. 3, although other types and/or numbers of equations, tables and/or other data to manage the condition including correlating treatment parameters may be stored by way of example. The application(s) can be implemented as modules or components of other applications. Further, the application(s) can be implemented as operating system extensions, module, plugins, or the like. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor 30, can be used for the memory 32.

The communication interface 34 of the programmable controller computing device 12 operatively couples and communicates between the programmable controller computing device 12 and the pressure cuff 14, the inflator 16, the pressure regulator 18, the pulse sensor 20, and/or the Doppler probe 21, although the communication interface 34 of the programmable controller computing device 12 can operatively couple and communicate with other types and/or numbers of other systems, devices, components, and/or other elements.

The display device 36 of the programmable controller computing device 12 may comprise one or more types of display devices, such as LCD or LED display screens or interactive touchscreens by way of example only. Additionally, the user input system 38 may comprise one or more user input devices, such as a keyboard, mouse, and/or interactive display screen by way of example only.

The examples for managing blood flow occlusion with the programmable controller computing device 12 may also be embodied as one or more non-transitory computer readable media having instructions stored thereon for one or more aspects of the present technology as described and illustrated by way of the examples herein. The instructions in some examples include executable code that, when executed by one or more processors, cause the processors to carry out steps necessary to implement the methods of the examples of this technology that are described and illustrated herein.

In this example, the pressure cuff 14 comprises a five (5) cm blood pressure cuff which may be used to facilitate controlled blood flow occlusion, although other types and/or numbers of other blood occlusion systems or devices may be used. Additionally, in this example the inflator 16 is a 50 psi/120 v inflator which may be coupled to and when engaged inflates the pressure cuff 14, although other types of systems and/or devices to control inflation and/or other operations of the pressure cuff 14 may be used.

The pressure regulator 18 is coupled to monitor and manage the pressure in the pressure cuff 14, although other types of regulation systems or devices to manage the pressure cuff may be used. In this example, the pressure regulator 18 comprises an electronic pneumatic pressure regulator, although other types of regulation systems and/or devices may be used.

The pulse sensor 20 is configured to measure and generate pulse data for the individual using the blood flow occlusion system 10, although other types and/or numbers of other sensors to manage the exemplary blood flow occlusion system 10 may be used.

The Doppler probe 21 is configured to measure and determine blood flow, such as radial flow by way of example only, although other types and/or numbers of systems or devices to measure blood flow and other manners for obtaining blood flow data may be used.

In this example, the power source 22 is coupled to supply power to the programmable controller computing device 12, the inflator 16, the pressure regulator 18, the pulse sensor 20, and the Doppler probe 21, although other types and/or numbers of power sources in other configurations may be used.

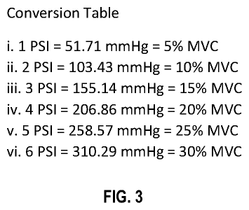

An exemplary method for managing a condition with the exemplary blood flow occlusion system 10 will now be described with reference to FIGS. 1-3. In step 200, this method starts when the power supply 22 is engaged and power is supplied to the programmable controller computing device 12, the inflator 16, the pressure regulator 18, the pulse sensor 20, and the Doppler probe 21 in this example, although other types of initialization steps may be used.

In step 202 the programmable controller computing device 12 generates and provides a graphical user interface on the display device 36 requesting entry with the user input system 38 of one or more diagnostic parameters to manage a condition, such as high blood pressure of client by way of example only. In this particular example, the input fields for the diagnostic parameters for managing a condition comprising high blood pressure are: the sex; humerus length; arm circumference; and resting blood pressure of the client with the condition to be managed by way of example only, although other types and/or number of other diagnostic parameters for managing the condition may be taken into account. For example, diagnostic parameters relating to the impact of race and/or ethnicity on the condition which may be correlated to stored treatment data by way of example could be used and/or factored into the occlusion regression equation, although other factors may be used. Additionally, in this particular example when entering the data for these values with the user input system 38 in the displayed input fields in the GUI on the display 36 in the programmable controller computing device 12, the value for the measurement of the arm in circumference is in cm, arm length is in cm, DBP and SBP are in mmHg and sex is (M-1, F-0), although other units can be used in other examples.

In step 204, the programmable controller computing device 12 receives the input values of the one or more diagnostic parameters in the input fields from the client using the user input system 38. The programmable controller computing device 12 may store these input values for the diagnostic parameters with an identifier for the client with the condition to be managed for future use in memory 32 or in other storage locations.

In step 206, the programmable controller computing device 12 may also engage the Doppler probe 21 to measure radial flow of the client to determine radial flow data which corresponds to radial occlusion data, although other types and/or numbers of measurements can be taken and the flow data and/or occlusion data can be obtained in other manners in other examples. In this particular example, the programmable controller computing device 12 is programmed to utilize the measured radial flow data of the client which provides radial occlusion data which corresponds to a reading of the brachial flow data which corresponds to measured brachial occlusion data, although the brachial flow data and/or occlusion data could be obtained in other manners in other examples.

In step 208, the programmable controller computing device 12 enters the input values for the diagnostic parameters for the current client in a stored occlusion regression equation to calculate brachial occlusion data. In this example, the programmable controller computing device 12 stores programmed instructions for executing a stored regression equation which in this example comprises: brachial occlusion=5 cm (R²=0.651 mmHg)=2.926 (arm circumference)+1.002 (bSBP)—0.428 (arm length)+0.213 (bDBP)+12.668 (sex)—68.493 for a 5 cm pressure cuff 14 and where 2.926, 1.002, 0.428, 0.213, 12.68 and −68.493 are the stored constants for this regression equation, although the programmable controller computing device 12 may store other equations with other determined constants for other cuff sizes and/or applications.

Next, because of the possibility of inaccuracies with the calculated brachial occlusion data and the measured brachial occlusion data, the programmable controller computing device 12 advantageously may generate optimized brachial occlusion data from the calculated brachial occlusion data and the measured brachial occlusion data, such as by taking an average of or using a median of the calculated brachial occlusion data and the measured brachial occlusion data by way of example only, although other data and/or manners for determining the brachial occlusion data can be used.

In step 210, the programmable controller computing device 12 determines a treatment program comprising treatment parameter data or other instructions to manage the condition of the client, in this example blood pressure, based on at least a correlation of the optimized brachial occlusion data and a current stage of the condition for the client, e.g. stage 1 or stage 2 high blood pressure, against one or more stored conversion tables and/or other stored treatment parameter data, such as the exemplary conversion table shown in FIG. 3, although manners for obtaining the treatment parameter data and other types of data can be correlated to obtain the treatment parameter data in other examples. By way of example, the treatment program for managing a high blood pressure condition of the client, may have correlated treatment parameters data from storage comprising a determined occlusion pressure to apply with the pressure cuff 14, time to maintain the occlusion pressure, a length of time for each cycle, a number of cycles, and time between cycles correlated to at least different optimized brachial occlusion data and different stages of the condition for the client. Accordingly, a sample treatment program may comprise calculated treatment parameter data of: five seconds inflation of pressure cuff 14 to a determined pressure of two PSI and then five seconds of deflation of the pressure cuff for three cycles of inflation and deflation.

In an illustrative example for a client with stage one hypertension and brachial occlusion data of 103.43 mmHg, the programmable controller computing device 12 may determine that the conversion table shown in FIG. 3 would identify two PSI as the desired pressure to apply during treatment. Additionally, the programmable controller computing device 12 would correlate the optimized brachial occlusion data, the stage of the condition and/or other data on the client, such as age, BMI, ethnicity, race, and/or other information (which in other examples may have correlated stored treatment parameter data) to determine a length of time for inflation at two PSI with the pressure cuff 14 on the arm of the client, the length of time the pressure cuff 14 is deflated and then number of cycles of this inflation and deflation.

In another example, for a client having a stage 2 hypertensive condition, the programmable controller computing device 12 may correlate and determine treatment parameter data for a treatment program comprising five cycles, at a pressure of four PSI per inflation (ten seconds on, ten seconds off) two times per day. The four PSI equates to 60% of occlusion (but can vary based on user parameters). In a further example, for the same client having a stage 1 hypertensive condition, the programmable controller computing device 12 may determine treatment parameter data for a treatment program that would be the same as above, except at five PSI in the pressure cuff 14, which equates to 80% occlusion. The programmable controller computing device 12 may also generate, in other examples, one or more additional variances with the treatment parameter data for a treatment program based on other factors, such as based on an analysis of the particular pathology and occlusion tolerance of the client requiring an adjustment, such as a percentage adjustment down, by way of example.

In this example, the various combinations of treatment parameter data for a treatment program are stored by the programmable controller computing device 12 in tables, such as the exemplary conversion table shown in FIG. 3, and/or other databases, although the treatment parameter can be stored in other storage locations or obtained in other manners. Additionally, in this example the stored conversion table in FIG. 3 provides a correlation for conversion of maximal voluntary contraction (MVC) conversion to PSI so that the exemplary blood flow occlusion system 10 is configured to mimic, as a passive system, a mechanical environment of isometric exercise on the client to treat the high blood pressure condition. With respect to the stage of the condition of the client which may be entered or retrieved by the programmable controller computing device 12 from stored medical records for the client, as the level of blood pressure of the client increases, then the amount of pressure applied would decrease, e.g. 80% occlusion if the measured blood pressure of the client is within stage one or 60% occlusion if the measured blood pressure of the client is within stage two by way of example.

The programmable controller computing device 12 also generates programmed instructions of control commands to execute the treatment program to manage operation of the pressure cuff 14, inflator 16, pressure regulator 18, pulse sensor 20, and/or Doppler probe 21 based on the generated treatment parameter data for the treatment program.

In step 212, the programmable controller computing device 12 initiates execution of the programmed instructions of control commands for the generated treatment parameter data for the treatment program for treating the condition of the client. In this example, the programmable controller computing device 12 energizes the inflator 16 to inflate the pressure cuff 14 around the arm of the client to the pressure set in accordance with the determined program. The pressure regulator 18 has a built-in pressure sensor that provides feedback to the programmable controller computing device 12 which uses this feedback to control the inflator 16 to inflate the pressure cuff 14 to the determined pressure and then disengage the inflator 16 unless needed to reengage the inflator 16 to maintain the determine pressure in the pressure cuff 14. The pressure regulator 18 is also configured with the programmable controller computing device 12 to automatically release any excess air from the pressure cuff 14 and prevent over inflation or injury to the client. If power is ever lost, the pneumatic regulator 18 also is configured to release all pressure and deflate the pressure cuff 14.

The programmable controller computing device 12 manages the pressure regulator 18 to maintain the inflated state at the determined pressure for the specified period of time in the determined program. Upon the completion of the inflation portion of the cycle, the programmable controller computing device 12 instructs the pressure regulator 18 to deflate the pressure cuff 14. The programmable controller computing device 12 maintains the deflated state for the pressure cuff for the desired time in the determined program. The programmable controller computing device 12 will repeat this inflation and deflation a number of cycles as set by the determined program.

The programmable controller computing device 12 may also receive and process pulse data from the pulse sensor 20 which is positioned on or otherwise coupled to the client to monitor the pulse of the client to, for example determine the effectiveness of the occlusion pressure and/or to ensure that a complete occlusion or other safety issue is not occurring. The pulse sensor 20 communicates with the programmable controller computing device 12 to determine when a pulse is detected and what the pulse is. When no pulse is detected by the pulse sensor 20, the programmable controller computing device 12 will adjust the pressure to a lower value to ensure complete occlusion is not occurring and may make other corrective adjustments as needed based on the detected pulse rate and a stored table of actions based on different pulse data readings.

Accordingly, as illustrated and described by way of the examples herein, this technology provides a blood flow occlusion system that effectively manages and treats high blood pressure. In particular, with the determined treatment program, examples of this technology are able to reduce resting blood pressure and treat hypertension of the client based on the decrease in blood supply and partial occlusion of the limb through passive, non-pharmacological means. Examples of this technology substantially increase accuracy of treating blood pressure by uniquely incorporating various correlated elements of data about the client to determine an effective program for occlusion to achieve the needed treatment benefit. Additionally, examples of this technology are also able to effectively monitor and avoid over occluding the vessel and damaging the surrounding vasculature of the client during treatment.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A blood flow occlusion system comprising:
a pressure cuff;
an inflator and a pressure regulator coupled to the pressure cuff; and
a programmable controller computing device coupled to the inflator and the pressure regulator and comprising a memory coupled to the processor which is configured to be capable of executing programmed instructions stored in the memory to:
determine brachial occlusion data based on calculating the brachial occlusion data with an occlusion regression equation using one or more input diagnostic parameters associated with a client;
generate one or more treatment parameters of a treatment program to treat the condition of the client based on at least the brachial occlusion data and an obtained stage of the condition for the client;
generate programmed instructions of control commands to manage operation of the pressure cuff, the inflator, and the pressure regulator based on the one or more treatment parameters of the treatment program; and
initiate execution of the programmed instructions of the control commands for the determined treatment program for treating the condition of the client when engaged.

2. The system as set forth in claim 1 wherein the memory coupled to the processor is further configured to be capable of executing programmed instructions stored in the memory to:
determine measured brachial occlusion data based on a blood flow reading of the client with the condition;
wherein the determine the brachial occlusion is further based on the calculated brachial occlusion data and the measured brachial occlusion data.

3. The system as set forth in claim 2 further comprising a Doppler probe coupled to the programmable controller computing device, wherein the blood flow reading is a Doppler radial blood flow reading of the client with the condition.

4. The system as set forth in claim 1 further comprising a pulse sensor coupled to the programmable controller computing device to provide a pulse reading of the client when engaged, wherein the memory coupled to the processor is further configured to be capable of executing programmed instructions stored in the memory to:
adjust operation of at least one of the control commands for at least one of the inflator or the pulse regulator based on the pulse reading of the client.

5. The system as set forth in claim 1 wherein the one or more diagnostic parameters comprise at least: a sex; a humerus length; an arm circumference; and a resting blood pressure of the client.

6. The system as set forth in claim 1 wherein the one or more treatment parameters of the treatment program to treat the condition of the client comprise: a set occlusion pressure, an inflation time of the pressure cuff, a deflation time of the pressure cuff, and the number of cycles of the inflation and the deflation.

7. A method for making a blood flow occlusion system, the method comprising:
providing a pressure cuff;
coupling an inflator and a pressure regulator to the pressure cuff; and
coupling a programmable controller computing device to the inflator and the pressure regulator and comprising a memory coupled to the processor which is configured to be capable of executing programmed instructions stored in the memory to:
determine brachial occlusion data based on calculating the brachial occlusion data with an occlusion regression equation using one or more input diagnostic parameters associated with a client;
generate one or more treatment parameters of a treatment program to treat the condition of the client based on at least the brachial occlusion data and an obtained stage of the condition for the client;
generate programmed instructions of control commands to manage operation of the pressure cuff, the inflator, and the pressure regulator based on the one or more treatment parameters of the treatment program; and
initiate execution of the programmed instructions of the control commands for the determined treatment program for treating the condition of the client when engaged.

8. The method as set forth in claim 7 wherein the memory coupled to the processor is further configured to be capable of executing programmed instructions stored in the memory to:
determine measured brachial occlusion data based on a blood flow reading of the client with the condition;
wherein the determine the brachial occlusion is further based on the calculated brachial occlusion data and the measured brachial occlusion data.

9. The method as set forth in claim 8 further comprising providing a Doppler probe coupled to the programmable controller computing device, wherein the blood flow reading is a Doppler radial blood flow reading of the client with the condition.

10. The method as set forth in claim 7 further comprising coupling a pulse sensor to the programmable controller computing device to provide a pulse reading of the client when engaged, wherein the memory coupled to the processor is further configured to be capable of executing programmed instructions stored in the memory to:
adjust operation of at least one of the control commands for at least one of the inflator or the pulse regulator based on the pulse reading of the client.

11. The method as set forth in claim 7 wherein the one or more diagnostic parameters comprise at least: a sex; a humerus length; an arm circumference; and a resting blood pressure of the client.

12. The method as set forth in claim 7 wherein the one or more treatment parameters of the treatment program to treat the condition of the client comprise: a set occlusion pressure, an inflation time of the pressure cuff, a deflation time of the pressure cuff, and the number of cycles of the inflation and the deflation.

13. A non-transitory computer readable medium having stored thereon instructions comprising executable code that, when executed by one or more processors, causes the one or more processors to:
determine brachial occlusion data based on calculating the brachial occlusion data with an occlusion regression equation using one or more input diagnostic parameters associated with a client;
generate one or more treatment parameters of a treatment program to treat the condition of the client based on at least the brachial occlusion data and an obtained stage of the condition for the client;
generate programmed instructions of control commands to manage operation of the pressure cuff, the inflator, and the pressure regulator based on the one or more treatment parameters of the treatment program; and
initiate execution of the programmed instructions of the control commands for the determined treatment program for treating the condition of the client when engaged.

14. The system as set forth in claim 13 wherein the instructions further comprise executable code that, when executed by one or more processors, causes the one or more processors to:
determine measured brachial occlusion data based on a blood flow reading of the client with the condition;
wherein the determine the brachial occlusion is further based on the calculated brachial occlusion data and the measured brachial occlusion data.

15. The non-transitory computer readable medium as set forth in claim 14 wherein the blood flow reading is a Doppler radial blood flow reading of the client with the condition.

16. The non-transitory computer readable medium as set forth in claim 13 wherein the instructions further comprise executable code that, when executed by one or more processors, causes the one or more processors to:
adjust operation of at least one of the control commands for at least one of the inflator or the pulse regulator based on the pulse reading of the client.

17. The non-transitory computer readable medium as set forth in claim 13 wherein the one or more diagnostic parameters comprise at least: a sex; a humerus length; an arm circumference; and a resting blood pressure of the client.

18. The non-transitory computer readable medium as set forth in claim 13 wherein the one or more treatment parameters of the treatment program to treat the condition of the client comprise: a set occlusion pressure, an inflation time of the pressure cuff, a deflation time of the pressure cuff, and the number of cycles of the inflation and the deflation
